(19) United States
(12) Patent Application Publication
(10) Pub. No.: US 2024/0366506 A1
(43) Pub. Date: Nov. 7, 2024

(54) ANTIMICROBIAL COATING MATERIAL AND METHODS FOR PREPARATION AND APPLICATION TO SURFACES

(71) Applicant: THE AMERICAN UNIVERSITY IN CAIRO, New Cairo (EG)

(72) Inventors: Mousa Elsayed Elsayed SALEM, Alexandria (EG); Wesam Awad Ahmed SARHAN, Madinaty (EG); Saif El-Din Mostafa Mamoon EL-MOFTY, Shubra El Kheima (EG); Noha Khalil Samy Aly MAHDY, Giza (EG); Abdalla Tarek Riad MOHAMED, Obour City (EG); Hassan M.E. AZZAZY, New Cairo (EG)

(73) Assignee: THE AMERICAN UNIVERSITY IN CAIRO, New Cairo (EG)

(21) Appl. No.: 18/690,519

(22) PCT Filed: Sep. 14, 2022
(86) PCT No.: PCT/US2022/043492

§ 371 Date: Mar. 8, 2024

Related U.S. Application Data
(60) Provisional application No. 63/243,952, filed on Sep. 14, 2021.

(51) Int. Cl. A61K 9/107; A61K 47/06

(52) U.S. Cl. CPC A61K 9/1075; A61K 47/06

(57) ABSTRACT

A composition comprising nanoparticles comprising benzoyl peroxide, benzyl chloride and/or benzalkonium chloride and a method using this composition to kill or inhibit the growth of microorganisms including bacteria and viruses.

### CROSS-REFERENCE TO A RELATED APPLICATION

This application claims priority to U.S. Provisional Application No. 63/243,952, filed Sep. 14, 2021 which is incorporated by reference for all purposes.

### BACKGROUND OF THE INVENTION

### Field of the Invention

The inventor pertains to the field of microbiology and medicine, and more specifically to an encapsulated form of benzoyl peroxide, benzyl chloride and/or benzalkonium chloride for inhibiting the growth or killing pathogenic microorganisms.

### Description of Related Art

Due to the COVID-19 pandemic and the continuing emergence of drug-resistant microorganisms increasing attention has been paid to the dangers of everyday exposure and contamination by microorganisms including both viruses, bacteria, fungi, protists, and parasites. The increasing virulence and transmission of these microorganisms has been primarily a concern for a wide range of healthcare facilities, and food processing and preparation facilities. However, it is also a concern for other locations especially high traffic locations including schools, markets and commercial facilities, offices and administrative facilities such as courts or a department of motor vehicles, public transport, airports, hotels, other businesses and the home. Healthcare facilities where microbial bio-burden is a concern include large multi-unit hospitals, specialized clinics, veteran affairs hospitals, long term care facilities, retirement homes and individual or group doctors or dental offices among others.

Spread and transmission of viruses like Influenza A, West Nile Virus, Zika virus MERS-CoV, and SARS-CoV (both SARS-CoV-1 and SARS-CoV-2) has been facilitated by increases in global travel and trade. New microorganisms, or more virulent forms of existing microorganisms, especially antibiotic resistant strains, are also being discovered, and can readily spread worldwide due to the growing ease of travel, and the developing worldwide market for goods.

Although regular cleaning and good sanitation practices can be is inherently resistant to, and can minimize the spread of microorganisms.

### BRIEF SUMMARY OF THE INVENTION

One aspect of the invention is directed to composition comprising benzoyl peroxide (“BPO”), benzyl chloride (“BC”), and/or benzalkonium chloride (“BAC”) encapsulated in nanoparticles of polymethylmethacrylate (PMMA). This composition is preferably dispersed as a spray, aerosol, or foam onto to an object or surface which may be contaminated or subject to contamination with a microorganism. Another aspect of the invention is a method for inhibiting the growth or replication of a microorganism or for inactivating or killing a microorganism by contacting it with, or by protecting an object or surface by application of, the nanoparticle composition disclosed herein.

This disclosure is also directed to a method for making the composition disclosed herein, for example, by (i) dissolving PMMA in a solute containing a surfactant, (ii) adding benzoyl peroxide or benzyl chloride to the dissolved PMMA, (iii) adding a non-solvent for PMMA to the solution for a time and under conditions suitable for nanofabrication of the PMMA around the benzoyl peroxide, benzylchloride or benzalkonium chloride.

One of the two preferred formulations is a nanoemulsion of PMMA loaded with BPO and/or BC. The second formulation comprises nanomicelles of BAC and BPO and/or BC. The preferred effective concentrations for the BPO, BC, and/or BAC are 0.1%, 0.2%, 0.3%, 0.4%, 0.5%, 0.6%, 0.7%, 0.8%, 0.9% to 1% (w/v); 0.1%, 0.2%, 0.3%, 0.4%, 0.5%, 0.6%, 0.7%, 0.8%, 0.9% to 1% (w/v), and 1%, 1.1%, 1.2%, 1.3%, 1.4%, 1.5%, 1.6%, 1.7%, 1.8%, 1.9% to 2% (w/v); respectively, based on the total volume of the composition or based on the total volume of the nanoparticles. In some embodiments, the concentration ranges described above are formulated on a w/w basis, w/v, or on a v/v basis.

In some embodiments, the nanoparticles encapsulate a mixture of two or more of BPO, BC and/or BAC (and/or other organic peroxides), for example, BPO+BC, BC+BAC, or BC+BAC. For mixtures of two of these, the relative ratios of BPO+BC, BC+BAC, or BC+BAC in the nanoparticles based on weight can range from <5:95, 10:90, 20:80, 30:70, 40:60, 50:50, 60:40, 70:30, 80:20, 90:10, 95:5 and >95:5 based on volume. When a third ingredient is present, it may be present in an amount of >0, 1, 2, 5, 10, 20, 30, 40, 50, 60, 70, 80, 90 or <100% based on volume or weight of the other two ingredients.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings below.

FIG. 1A graphically illustrates a general process for producing a nanoemulsion.

FIG. 1B graphically illustrates micelle formation from amphiphiles.

FIG. 1C describes a process nanoemulsion synthesis.

FIG. 1D describes a process for nanomicelles synthesis.

FIG. 2A depicts an SEM image the structure of nanomicelles.

FIG. 2B depicts an SEM image the structure of nanomicelles.

### DETAILED DESCRIPTION OF THE INVENTION

The COVID-19 global pandemic and other emerging pathogens such as Monkeypox virus have highlighted the great demand for antimicrobial/antiviral coatings to suppress the transmission of the virus. Over the last decade, several reports described the introduction of antimicrobial/antiviral coatings that can be applied to different surfaces. However, their widespread application is still not realized, especially in Egypt. In view of the COVID-19 pandemic, the inventor sought to develop a surface coating that is easily applied and effective against a broad range of microbial pathogens, including bacteria and SARS-CoV-2 and other viruses. In one embodiment, this surface coating is designated NanoGuard. Preferably, this composition as a spray, for example, onto surfaces, equipment, tools or other objections in hospitals, schools, universities, and homes to eliminate microbial communities and prevent disease transmission. Such a spray deposits a layer with sustained antimicrobial/antiviral effect which can last for several days, thus significantly reducing the cost of disinfection, reducing transmission of a microorganism, and reducing the development of resistance by a cidal effect on the microorganism.

Benzoyl peroxide, benzyl chloride and benzalkonium chloride exhibit a versatile mode of actions against microbes hence rendering them insensitive to development of bacterial or microbial resistance. The NanoGuard surface coating represents an innovative, cost effective, safe technology via nanoencapsulation of benzoyl peroxide (“BPO”) radical-generating compound, benzyl chloride (“BC”) and/or benzalkonium chloride (“BAC”) into polymethylmethacrylate (PMMA) adhesive nanoparticles. The nanoparticles contained in the compositions disclosed herein can maintain prolonged release of the free radicals and increased adhesion to surfaces thus prolong antiviral/antibacterial/antimicrobial activity to several days. The composition disclosed herein may be applied to surface exposed to, or containing the following non-limited list of bacteria: Bacillus anthracis Bordetella pertussis, Borrelia burgdorferi, Brucella abortus, Brucella canis, Brucella melitensis, Brucella suis, Campylobacter jejuni, Chlamydia pneumoniae, Chlamydia psittaci, Chlamydia trachomatis, Clostridium botulinum, Clostridium difficile, Clostridium perfringens, Clostridium tetani, Corynebacterium diphtheria, Enterococcus faecalis, Enterococcus faecium, Escherichia coli, Enterotoxigenic Escherichia coli (ETEC), Enteropathogenic E. coli, E. coli (O157:H7), Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitides, Pseudomonas aeruginosa, Rickettsia rickettsia, Salmonella typhi, Salmonella typhimurium, Shigella sonnei, Staphylococcus aureusa, Staphylococcus epidermidis, Staphylococcus saprophyticus, Streptococcus agalactiae, Streptococcus pneumoniae, Streptococcus pyogenes, Treponema pallidum, Vibrio cholorae and Yersinia pestis.

It may be used to control, inhibit or kill microorganisms causing or contributing to nosocomial infections including, methicillin-resistant Staphylococcus aureus (MRSA), viruses, bacterial or other microorganisms causing or contributing to acute or chronic inflammation of the lungs, (pneumonia) including microorganisms associated ventilator-associated pneumonia or with hospital-acquired pneumonia (nosocomial pneumonia). It may be used to prevent or control infections by Staphylococcus aureus, Methicillin resistant Staphylococcus aureus, Candida albicans, Pseudomonas aeruginosa, Acinetobacter baumannii, Stenotrophomonas maltophilia, or Clostridium difficile; of those microorganisms causing or associated with tuberculosis, urinary tract infection, hospital-acquired pneumonia, gastroenteritis, vancomycin-resistant Enterococcus, or Legionnaires' disease.

The composition disclosed herein may be applied to surface exposed to, or containing the following non-limited list of viruses: Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Coronavirus (including COVID-19, SARS or MERS), Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirusalitis, GB virus C/Hepatitis G virus Pegivirus, Hantaan virus, Hendra virus, Hepatitis A virus, Hepatitis B virus, Hepatitis C virus, Hepatitis E virus, Hepatitis delta virus, Horsepox virus, Monkeypox virus, Camelpox virus, Orthopoxvirus, Parapoxyvirus, Vatapoxvirus, and Mollusciipoxvirus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus O, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus.

The composition and method disclosed herein may also be used to sanitize or sterilize objects or surfaces contaminated with other animal or plant viruses. In some embodiments, treatment of a surface will remove, inactivate or kill 100%, <100%, 99%, 98%, 97%, 96%, 905%, 90%, 85%, 80%, 75%, 70%, 65%, 60%, 55%, 50% or <50% of one or more types of microorganisms.

The disclosed composition may contain benzoyl peroxide, benzyl chloride and/or benzalkonium chloride in a form for immediate or sustained release once applied to an external object or surface.

Benzoylperoxide is an organic peroxide with the structural formula often abbreviated as (BzO)₂ or BPO:

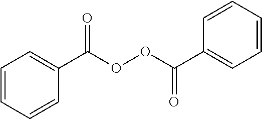 [SMILES: O=C(OOC(=O)c1ccccc1)c1ccccc1]

BPO is a white granular solid with a faint odor of benzaldehyde, poorly soluble in water but soluble in acetone, ethanol, and many other organic solvents. Benzoyl peroxide is an oxidizer, which is principally used as in the production of polymers. As a medication, benzoyl peroxide is mostly used to treat acne, either alone or in combination with other treatments and is also used in dentistry for teeth whitening. Benzoyl peroxide thermally degrades to benzoic acid and free oxygen radicals. The former lowers pH on a contacted object or surface, the latter disrupts microbial cell membranes. The oxygen radicals propagate the reaction by reacting with BPO or a solvent to form other benzoate or solvent radicals.

Benzoyl chloride, also known as benzenecarbonyl chloride, is an organochlorine compound with the formula C₇H₅ClO. It is a colorless, fuming liquid with an irritating odor. It is mainly useful for the production of peroxides but is generally useful in other areas such as in the preparation of dyes, perfumes, pharmaceuticals, and resins. It reacts with water to produce hydrochloric acid and benzoic acid:

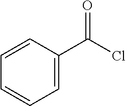

Benzalkonium chloride (BAC) (CAS Number 8001-54-5) is a type of cationic surfactant. It is an organic salt classified as a quaternary ammonium compound. BACs are a mixture of alkylbenzyldimethylammonium chlorides, in which the alkyl group has various even-numbered alkyl chain lengths.

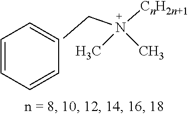

Organoperoxides. In some alternative embodiments, an organic peroxide other than BPO may be incorporated into a nanoparticle as disclosed herein. These include hydroperoxides, compounds with the functionality ROOH (R=alkyl, for example C₁-C₆ alkyl); peroxy acids and esters, compounds with the functionality RC(O)OOH and RC(O)OOR′ (R,R′=alkyl, aryl, for example, C₁-C₆ alkyl); diacyl peroxides, compounds with the functionality RC(O)OOC(O)R (R=alkyl, aryl, for example C₁-C₆ alkyl); or dialkylperoxides, compounds with the functionality ROOR (R=alkyl, for example C₁-C₆ alkyl). The term C₁-C₆ alkyl as used herein includes C₁, C₂, C₃, C₄, C₅ and C₆ alkyl including linear or branched alkyl.

The inventor considers that the antimicrobial activity provided by the release of BPO, BC and/or BAC or other organoperoxides released from the nanoparticles disclosed herein will persist for at least 1, 2, 4, 6, 8, 12, 24 or 48 hours and that this antimicrobial activity is effective against a broad spectrum of microbes.

Nanoparticles. The term “nanoparticles” or “nanocapsules” refers to particles comprising BPO, BC and/or BAC which are mixed with, or substantially uniformly mixed with, the PMMA. This term includes micro- or nanomicelles containing these ingredients.

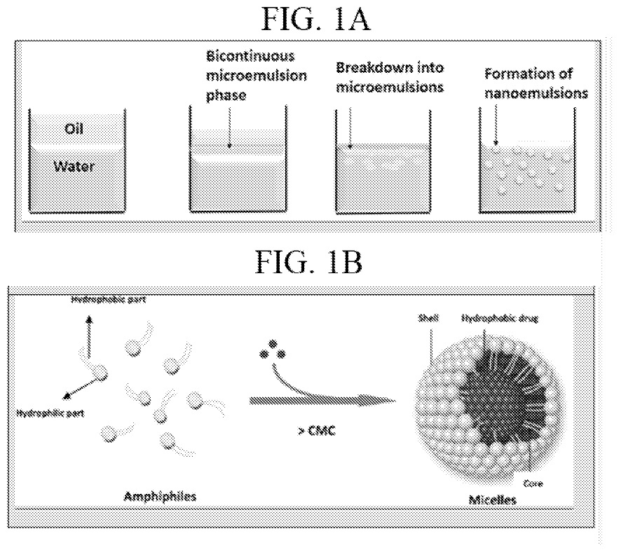

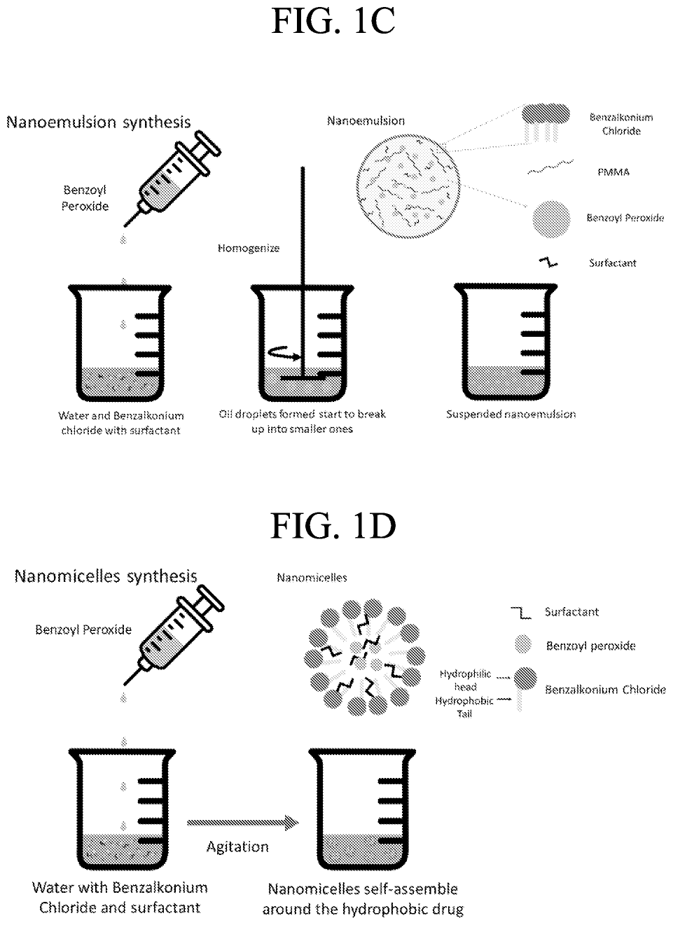

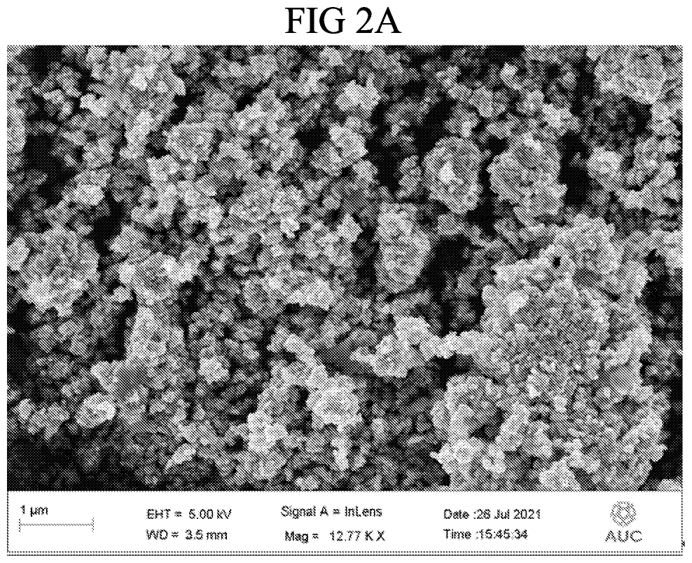

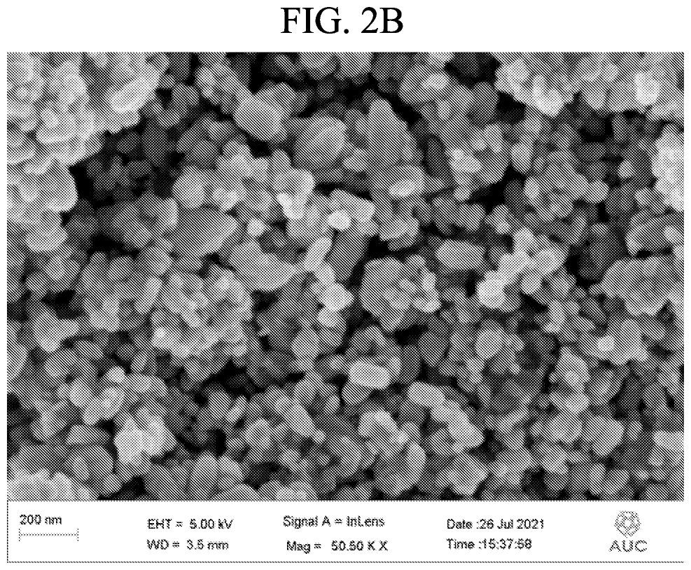

Nanomicelles are ultramicrosopic globular structures that typically consist of exterior hydrophilic polar heads and an interior hydrophobic fatty acyl chain. FIG. 1C describes a method for producing a nanoemulsion. Nanomicelles and a mode of their synthesis are described in FIG. 1D. Preferably, nanomicelles encompass a nanocarrier comprising BPO, BC and/or BAC as active ingredients which form the nanomicelles.

The nanoformulations are synthesized in three forms: The first one comprises nanoparticles that comprise the PMMA polymeric matrix with BC or/and BPO embedded within its matrix which is stabilized and coated with the BAC. The second one comprises PMMA nanoemulsion loaded with BPO or/and BC within its core and is stabilized and coated with BAC. The third one comprises nanomicelles that are formed of the active ingredients alone without the PMMA polymeric carrier. Preferably, the nanomicelles are formed of BPO or/and BC as well as BAC only in addition to a suitable surfactant.

Typically, the average diameter of the nanomicelles ranges from 1, 2, 5, 10, 20, 50, 100, 200, 500, 1,000 to 2,000 nm, preferably, about 50 to 500 nm. The ranges disclosed herein include the range endpoints as well as intermediate values and subranges. Thus, size ranges from 20, 50, 100, 200, 500 to 1000 nm are effective when the previously mentioned criteria are maintained.

The concentration of BPO, BC and/or BAC in the nanomicelles ranges from <0.01, 0.01, 0.02, 0.05, 0.1, 0.2, 0.5, 1, 2, 5 or >5 wt % or vol. %. The preferred concentration ranges of the active ingredients are: 0.1%, 0.2%, 0.5% to 1% (w/v) for the BPO, 1%, 1.2%, 1.5%, to 2% (w/v) for BAC, and 0.1%, 0.2%, 0.5% to 1% (w/v) for the BC. In some embodiments, the concentration ranges described above are formulated on a w/w basis, w/v, or on a v/v basis.

Poly(methylmethacrylate) (PMMA) also known as acrylic, acrylic glass, or plexiglass, as well as by the trade names Crylux®, Plexiglas®, Acrylite®, Astariglas®, Lucite®, Percla®, and Perspex®, among several others is a transparent thermoplastic often used in sheet form as a lightweight or shatter-resistant alternative to glass. As disclosed herein, PMMA is produced in a form suitable for encapsulating BPO, BC and/or BAC. A constituent monomer of PMMA is shown below. The preferred average MW for the PMMA are the small and medium molecular weights of the following averages; 3.5, 4, 5, 10, 20, 30, 40, to 50 kDa for the small molecular weight and average 100, 110, 120, 130, 140, 150 kDa, preferably about 100, 110, 120, 130 to 140 kDa for the medium molecular weight.

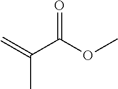

PMMA co-monomers. In some embodiments, 0, >0, 5, 10, 20, 30, 40, 50, 60, 70, 80, 90, 95 or >95% wt % of a co-monomer of PMMA, such as butylacrylate may be incorporated into a PMMA and benzoyl peroxide composition as disclosed herein.

In some embodiments, one or more of the following materials may be mixed in the same or similar content ranges with PMMA in combination with, or exclusive of, butylacrylate.

Poly(methyl acrylate. The polymer of methyl acrylate, PMA or poly(methyl acrylate), is similar to poly(methyl methacrylate), except for the lack of methyl groups on the backbone carbon chain. PMA is a soft white rubbery material that is softer than PMMA because its long polymer chains are thinner and smoother and can more easily slide past each other. In some embodiments, 0, >0, 5, 10, 20, 30, 40, 50, 60, 70, 80, 90, 95 or >95% wt % of PMA may be incorporated into a PMMA and benzoyl peroxide nanoparticles as disclosed herein.

Poly(ethyl methacrylate) (PEMA). The polymer of ethyl methacrylate, is similar to poly(methyl methacrylate), except for methyl groups which are alternated with ethyl groups in the constituting monomer. PEMA has an overall softer texture than PMMA as it has a lower modulus of elasticity. In some embodiments, 0, >0, 5, 10, 20, 30, 40, 50, 60, 70, 80, 90, 95 or >95% wt % of PEMA may be incorporated into a PMMA and benzoyl peroxide nanoparticles as disclosed herein.

Poly(hydroxyethyl) methacrylate (PHEMA). The polymer of hydroxyethyl methacrylate backbone monomer. PHEMA is optically transparent stable hydrophobic polymer. In some embodiments, 0, >0, 5, 10, 20, 30, 40, 50, 60, 70, 80, 90, 95 or >95% wt % of PEMA may be incorporated into a PMMA and benzoyl peroxide nanoparticles as disclosed herein.

Other polymers that may be used, or used in combination with PMMA, to form the coating or shell of the nanoparticles disclosed herein include, but are not limited to, Polypropylcyanoacrylate, Polylactic co-glyconic acid (PLGA), Sulfobutylated polyvinylalcohol-PLGA, Lectin-PLGA, Gliadin, Lectin-Gliadin, Chitosan Polyethyleneglycolpolylactic acid, Polymethylvinylether-co-maleic anhydride, Polyethylene oxide-polyoxypropylene (PEO-POP), Poly N-isopropylacrylamide, Poly N-vinylacetamide, Poly t-butylmethacrylate, Polycaprolactone (PCL), Gelatin (plant or animal based), Polystyren, Hydroxypropylmethylcellulose phthalate (HPMCP 50), Hydroxypropylmethylcellulose Ophthalate (HPMCP 55) Methacrylic acid/Ethylacrylate co-polymers: Eudragit S 100* (anionic polymer, and Eudragit E 100** polymer. In some embodiments, 0, >0, 5, 10, 20, 30, 40, 50 or >50 wt % of PEMA may be incorporated into a PMMA and benzoyl peroxide nanoparticles as disclosed herein.

Nanoencapsulation. A suitable micro- or nanoencapsulation technique and encapsulating BPO, BC and/or BAC may be selected by one with skill in the art. Such techniques and materials are also described by and incorporated by reference to Suganya, V. et al., Microencapsulation and Nanoencapsulation: A Review INTERNATIONAL JOURNAL OF PHARMACEUTICAL AND CLINICAL RESEARCH 2017; 9(3): 233-239.

Process for making BPO, BC and/or BAC containing-PMMA nanoparticles. In a preferred embodiment, the nanoparticles as disclosed herein are made (a) forming an emulsion comprising BPO, BC and/or BAC, and (b) forming coated nanoparticles from said emulsion, wherein said nanoparticles have an average particle size within the range of 20-2500 nm.

In one embodiment, nanoparticles of water-soluble and water-insoluble substances by controlled precipitation, co-precipitation and self-organization processes in a microjet reactor of colliding jet streams of agents. In a first jet stream, a solvent containing at least one target molecule collides with a second jet stream of a nonsolvent. As the jets collide with each other in the microjet reactor at pressures and flow rates that form turbulent mixing conditions, the target molecule becomes enveloped in the coating material and rapidly precipitates, co-precipitates or a chemical reacts thereby forming microparticles or nanoparticles.

The particle size be controlled by the temperature at which the solvent and nonsolvent collide, the flow rates of the solvent and the nonsolvent, and/or the amount of gas in the microreactor. Smaller particle sizes are generally obtained at lower temperatures, high solvent and nonsolvent flow rates, and/or in the complete absence of gas.

The disclosed method produces nanoparticles of water-soluble and water-insoluble substances by controlled precipitation, co-precipitation and self-organization processes in microjet reactors in which a first microjet of solvent containing at least one target molecule collides with a second microjet of a nonsolvent in a microjet reactor volume at a defined pressure and flow rate. The colliding jets effect very rapid precipitation, co-precipitation, or a chemical reaction during which the desired microparticles or nanoparticles are formed in a suspension of the particulate product in a mix of solvent and nonsolvent. The solvent and nonsolvent are removed by evaporation thereby leaving the desired particulate product.

The desired particle size range of the particulate product from the present process is small with a narrow distribution. The size and distribution are controlled by the temperature at which the solvent and nonsolvent collide, the flow rates of the solvent and the nonsolvent, and/or the amount of gas.

The first microjet contains a solvent for the target molecule of interest and any additional components or auxiliary components that should be found in the nanoparticle product. In the present case, that target molecule includes the BPO or other peroxide, BC and/or BAC. In one embodiment, the first microjet stream contains only the BPO, BC and/or BAC and a solvent with no other additives or ingredients. In another embodiment, the first microjet stream contains one or more ingredients used to form the coating that encapsulates the BPO, BC and/or BAC or other peroxide and any stabilizing agents or surfactants. In a further embodiment, the first microjet contains all of the ingredients in solution that will spontaneously form the desired nanoparticles upon contact with the nonsolvent in the second microjet stream.

Suitable solvents for BPO, BC and/or BAC are organic, e.g. one, two or more of tetrahydrofuran (THF), ethanol, acetone, or a mixture of acetone:ethanol 50/50 (v/v). A preferred solvent formulation contains a mixture of ethanol, polysorbate, polyoxyethylene sorbitan monopalmitate (Tween 20), and polycaprolactone.

A second method of nanoparticles synthesis, the emulsion-solvent evaporation method, is used to encapsulate hydrophilic or hydrophobic drugs in nano- or micro-particles or nano- or micro-micelles. Nanoparticles of few hundred nanometer in diameter can be obtained by precipitation of polymer encapsulating the drug once the evaporation of the organic phase of a nanoemulsion has taken place. The prepared nanoemulsion may be a single emulsion, oil-in-water (O/W) or water-in-oil (W/O), or a double emulsion; water-in-oil-in-water (W/O/W). Typically, the type of emulsion method depends on solubility of the drug, properties of the polymer and the degree of miscibility of solvent (organic, oil) with water phase. A nanoemulsion is prepared by addition of an immiscible solvent to another one (in a considerably larger volume ratio) during a high shear mixing and in presence of a surfactant to overcome the surface tension between the two immiscible solvent encouraging the production of a well-dispersed emulsion.

The particle size of the nanoparticles may be tuned via modifications in the temperature of the solvents during addition, the rate of addition of the internal phase solvent, the shear rate of the external phase during addition, the type of the surfactant used (in regards with its chemical structure and its hydrophilic-lipophilic balance (HLB), the volume ratio of the two immiscible solvents, and/or the concentrations of each constituent in the formulation.

In the O/W emulsion-solvent technique the organic phase is formed by dissolving the hydrophobic substance (the BPO and a polymer such as, polylactic acid (PLA), Polymethyl methacrylate (PMMA), polylacto-coglycolic acid (PLGA)) in a volatile water immiscible organic solvent as ethyl acetate, dichlromethane, ethyl ether, or chloroform. Predominantly, the aqueous phase contain a surfactant like poly(vinyl alcohol) (PVA), tween, sodium dodecyl sulfate, triton-X100, benzalkonium chloride, or pluronic. Subsequently, the emulsion is prepared by adding organic phase to aqueous phase drop wise during a very high shear mixing. Afterwards, the organic solvent is permitted to evaporate by overnight constant stirring at room temperature. Worth mentioning, the NPs are recovered by ultracentrifugation and washing with distilled water to get rid of excess of surfactant and free drug. Distilled water is subsequently removed by lyophilization to extend shelf life time during NPs storage, after addition of a cryoprotectant to minimize the undesired effects of lyophilization on the NPs.

The third technique used is the nanomicelles formation, which rely on the spontaneous formation of micelles after the addition of an organic solvent to another bulk solution in the presence of a surfactant with a concentration beyond its critical micelle concentration (CMC). Preparation of Nanomicelles (NMs) was considered as it is expected to be very cost effective, as: the use of a polymer can be avoided, and the technique is less hectic; no high-shear mixing is required during addition of the internal organic phase to the external aqueous phase.

As via the previously mentioned technique, also using the nanomicelles formation, the particle size of the nanoparticles can be modified by changing the temperature of the solvents during addition, the rate of addition of the organic phase solvent, the shear rate of the external aqueous phase during addition, the type of the surfactant used (in regards with its chemical structure and its hydrophilic-lipophilic balance (HLB), the volume ratio of the two miscible solvents, and/or the concentrations of each constituent in the formulation.

Here, the internal organic phase (like acetone, ethanol, or a mixture of both) with dissolved substances to be encapsulated will be added to the external phase during moderate stirring. After than the organic phase is evaporated by overnight continuous stirring.

The preferred solvents are: chloroform, dichloromethane, ethyl ether, ethyl acetate, acetone, and ethanol which are compatible with BPO, BC, and BAC.

The second microjet contains a nonsolvent, optionally containing one or more auxiliary agents. Suitable non-solvents include water, polysorbents and stabilizing agents such as poloxamers 127, 182, 184, 188, 338, 401, 402 or 407 or poloxamines 904, 908, 1107 or 1307 the concentrations of which depend on the dissolution characteristics and the partition coefficient (log P) between the aqueous and lipid phase of the active pharmaceutical ingredient developed in a nanoparticle dosage format.

Poloxamers are nonionic triblock copolymers composed of a central hydrophobic chain of polyoxypropylene (poly(propylene oxide)) flanked by two hydrophilic chains of polyoxyethylene (poly(ethylene oxide)). For the generic term poloxamer, these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g. P407=poloxamer with a polyoxypropylene molecular mass of 4000 g/mol and a 70% polyoxyethylene content). The preferred second microjet contains primarily water.

The weight ratio of target molecule in the first microjet stream to any auxiliary agent in the second microjet stream is preferably at least 1:100, more preferably within the range of 1:100 to 1:1,000; 1:1,000 to 1:1,000 to 1:10,000; 1:10,000 to 1:100,000, and even more preferably within the range of 1:200-1:75,000; or 1:2,000 to 1:7,500.

The microjet reactor used in the present process has at least two nozzles oriented to impinge their respective flow streams at an angle within the range of 90° to 180° relative to the other. Each nozzle has its own pump and feed line for injecting one liquid medium into a central reactor chamber within a reactor housing to a shared collision point. The reactor housing can be provided with a first opening through which a gas can be introduced so as to maintain the gaseous atmosphere within the reactor chamber at the collision point of the liquid jets. The gas line can also help to cool the resulting products. An exhaust opening is also provided that provides an exit for the resulting products and any excess gas. The reactor housing is preferably submerged and substantially completely surrounded by a water bath that can help to control the temperature within the reactor.

It is possible to use two microjet reactors in series to provide the nanoparticles produced in the first reactor with an external coating or adhesive in the second reactor. The resulting coated nanoparticle may then be adjusted for the desired release timing after deposit.

The temperature at which the liquids collide is one mechanism for control of the resulting particle size. Lower temperatures lead to decreasing particle sizes. In general, a desirable temperature is within the range of 25° to 95° C., preferably within the range of 40° to 80° C., and even more preferably within the range of 50-75° C.

Smaller particle sizes are also obtained by reducing the amount of gas, right through to a complete absence thereof, in the reactor chamber.

Preferably, there is little air or inert gas in the collision chamber. Increasing amounts of air can influence interactions between the developing diffusion layers such that, in many applications, relatively large nanoparticles are ultimately formed and can lead to undesired crystal growth. Conversely, it was found that a complete absence of air or inert gas led to the formation of smaller particles of a narrower size distribution. If no added stream of gas is used, the rapid precipitation of particles ends as soon as the liquid jets reach the outer periphery of the liquid disc formed when they collide as impinging jets. This presumably results in early termination of particle growth and in smaller particles showing homogeneous particle distribution.

Particle size may also be controlled via solvent and nonsolvent flow rates. Namely, smaller particles are obtained by selecting a high flow rate while larger particles result when selecting a low flow rate.

The solvent and non-solvent streams preferably each independently have a flow rate of greater than 0.1 ml/min and produce impinging jets that collide at a relative speed of greater than 25 m/s, more preferably within the range of 50-350 m/s with a Reynolds number at collision of more than 100, preferably more than 500, to obtain turbulent, rapid mixing at the collision point. A useful molar range of BPO, BC and/or BAC to nonsolvent is within the range of 0.1, 0.2, 0.5, 1, 2, 5, 10, 20, 50-100.

The solvent and nonsolvent nozzles are preferably smaller than 1,000 μm, more preferably smaller than 500 μm, and highly preferred to have an opening of smaller than 300 μm and have a driving pressure of at least 0.2 bar, preferably within the range of 0.5-100 bar and even more preferably within the range of 0.75-25 bar, the pressure being controlled by a pressure regulator associated with the feed stream to each of the first and second microjet nozzles.

The resulting coating is insoluble in water but capable of release of BPO, BC and/or BAC when applied to an object or surface. Insolubility in water allows the coated nanoparticles to be added to foods and liquids without storage degradation or release of odors. The coating is degradable or permeable so that the nanoparticles to break down or release BPO, BC and/or BAC in a controlled manner after application to a surface to be sanitized or sterilized. Preferably, the molar ratio of polymeric coating to the sum of all encapsulated BPO, BC and/or BAC is within the range of <0.5, 0.5, 1, 2, 10, 20, 50, 100, 200, 500, 100, 2000, 5000, to >5000.

In some embodiments, the nanoparticles are formulated to release an antimicrobial concentration of BPO, BC and/or BAC over a period of at least 4, 6, 8, 12, 16, 20, 24, 36 or 48 hours. In some embodiments, an antimicrobial concentration of BPO, BC and/or BAC is released at a release rate of at least 1, 2, 3, 4, 5, 6, 7, 8, 9, 10 wt % of the BPO, BC and/or BAC contained in a nanoparticle over a period of 1 hour. Release of the BPO, BC and/or BAC may also be substantially exponential and decline by a factor of 2 over a period of 1, 2, 3, 4, 5, 6, 7, 8, 9 or 10 hours.

In the event that two polymers (e.g., Polymer A and Polymer B) are used to form the encapsulation coating, the molar ratio of Polymer A to Polymer B is preferably within the range of 1:200 to 200:1, 1:100 to 100:1, 1:50 to 50:1, 1:25 to 25:1, 10:1 to 1:10, 1:5 or 5:1 or 1:2 to 2:1 with each polymer solution exhibiting a concentration within the range of 0.01 mg/mL to 10 mg/mL. In some embodiments, Polymer A and Polymer B may form separate concentric coatings.

The resulting coated nanoparticles of the present process exhibit an average particle size within the range of 20, 50, 100, 200, 500, 600, 700, 800, 900, 1,000, 1100, 1200, 1300, 1400, 1500, 1600, 1700, 1800, 1900 or 2,000-2500 nm. Preferably, the coated nanoparticles have an average size within the range of 25, 50, 100, 200, 500-1000 nm, and especially an average size within the range of 100, 200, 300, 400 to 500 nm, and are sufficiently small to be suspended in beverages without discernible notice or detection by the consumer.

The particle size distribution of coated BPO, BC and/or BAC made according to the invention have a narrow particle size distribution that is believed to enhance the uniform rate of release of BPO, BC and/or BAC. A suitable average particle size for the encapsulated BPO, BA and/or BAC is within the range from about 25-2500 nm, preferably an average particle size within the range of 25, 50, 100, 200, 300, 400, 500, 600, 700, 800, 900 to 1,000 nm, and an especially preferred particle size distribution is an average particle size within the range of 100, 200, 300, or 400-500 nm.

The polydispersity index (PDI) of the generated particles is generally no more than 2.0, preferably no more than 1.0, more preferably no more than 0.5, and most preferably no more than 0.4. This makes for an optimal size for bioavailability and treatment according to the invention. In physical and organic chemistry, the dispersity is a measure of the heterogeneity of sizes of molecules or particles in a mixture. A collection of objects is called uniform if the objects have the same size, shape, or mass. A sample of objects that have an inconsistent size, shape and mass distribution is called non-uniform. The polydispersity index uses dynamic light scattering under the cumulant method to measure the size distribution variance profile of small particles (no more than 250 nm) in suspension. With the dynamic light scattering measurement technique, the dispersity values of small particles in suspension are generally in the range from 0 (uniform size) to 1 (non-uniform size).

For easier redispersibility of the prepared nanoparticles, the polydispersity index (PDI) should be as low as possible indicating a narrow particle size distribution. This will lead to a minimized segregation, hence a minimized caking and maximized redispersibility of the NPs. The preferred PDI of the BPO, BC and/or BAC containing nanoparticles is less than or equal to 0.1, 0.15, 0.2, 0.25, or 0.3 with a smaller PDI being preferred.

Highly charged NPs, whether negatively charged or positively charged, are preferred to enhance the repulsion between the NPs, reducing their aggregation which improve their redispersibility. The preferred zeta potential of the BPO containing nanoparticles is more than 10, 15, 20, 25 or 30 mV (larger zeta potential is better).

Rate of diffusion. The present disclosure provides methods for producing suspendable nanoparticles containing BPO, BC and/or BAC that can be applied to objects and surfaces to sanitize, reduce the number of viable or infectious microorganisms, or sterilize them by the action of BPO, BC and/or BAC. The invention provides nanoparticles containing diffusible active ingredients which can prolong the exposure of an object or surface to these active ingredients. The rate of diffusion of these active ingredients out of a formed nanoparticle may be selected by increasing or decreasing the average nanoparticle size, the regularity shape of the nanoparticles, concentrations of active ingredients in the encapsulated nanoparticles, or the formulation of the encapsulating medium, such as the concentration or polymer chain length of PMMA, the natural polymer viscosity of the PMMA, the degradation rate of PMMA, the viscosity of the matrix of which the active ingredient will be release into, the solubility of active ingredient in that matrix, the concentration gradient of the active ingredient to the matrix applied on an object or surface.

Coatings/Adhesiveness. In some embodiments, the nanoparticles as disclosed herein are coated with one or more polymers that stabilize the particle against premature degradation or which provide adhesiveness to an object or surface to which they are applied. The process of coating of the formed BPO, BC and/or BAC nanoparticles is made during a one-step formation process in which the BPO, BC and/or BAC in a liquid form at the appropriate rate are dissolved in one or more organic solvents to which are added coating agents, such as plant or animal-based gelatin, synthetic or natural gum derivatives, e.g. arabic gum or pectin. In some embodiments, the nanoparticles are formulated to bind to microbial membranes. The nanoparticles are ultimately coated with PMMA which is known as a widely used biomaterial in contact lenses and other biomedical applications, it has high adherence capabilities both to microbes and different solid surfaces.

Suitable polymers that can be used as such coatings, their respective concentrations as introduced into the microjet chamber and impacted as directed by the present specification. The result is a suspension of coated or encapsulated particulates containing BPO, BC and/or BAC at the ratio of the incoming stream with a small size (e.g., 100-500 nm) and very narrow particle size distribution, e.g., a PDI or less than about 1, desirably less than about 0.75, more desirably within the range of with polydispersity index within the range of 0.001 to less than 0.25, preferably within the range of 0.005-0.20, and especially preferred of a PDI within the range of 0.005-0.15 at a 95% confidence interval. Such a narrow particle size distribution of nanoparticles provides an uniform dispersion of BPO at a high potency.

Preferably, the molar ratio of polymeric coating to containing BPO, BC and/or BAC is within the range of 0.5, 1, 2, 5, 10, 20, 50, 100, 200, 500, 1000, 2000, 3000, 4000, to 5000. In the event that two different polymers are used to coating the core, the molar ratio of the first polymer to the second polymer is preferably within the range of 1:200 to 200:1 with the first and second polymers each independently having a concentration within the range of 0.01, 0.02, 0.05, 0.1, 0.2, 0.5, 1, 2, 5 or 10 mg/ml or any intermediate subrange or value.

If desired, the nanoparticles or coated nanoparticles of the present invention can include a pH buffer to selectively adjust the pH from <3, 3, 4, 5, 6, 7, 8, 9, 10 or >10 to facilitate antimicrobial activity. An increase in pH improves the antimicrobial activity of some disinfectants (e.g., glutaraldehyde, quaternary ammonium compounds) but decreases the antimicrobial activity of others (e.g., phenols, hypochlorites, and iodine). The pH influences the antimicrobial activity by altering the disinfectant molecule or the cell surface.

Carriers & Excipients. The compositions can be dispensed alone or more typically in combination with a conventional pharmaceutical carrier or excipient, excipient or the like. The term “excipient” is used herein to describe any ingredient other than the nanoparticles disclosed herein. Pharmaceutically acceptable excipients include, but are not limited to, ion exchangers, alumina, aluminum stearate, lecithin, surfactants used in pharmaceutical dosage forms such as Tweens, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β- and γ-cyclodextrin, or chemically modified derivatives such as hydroxyalkylcyclodextrins, including 2- and 3-hydroxypropyl-b-cyclodextrins, or other solubilized derivatives can also be advantageously used to enhance delivery of compositions described herein. Actual methods of preparing such dosage forms are known, or will be apparent, to those skilled in this art; for example, see REMINGTON: THE SCIENCE AND PRACTICE OF PHARMACY, 21st Edition (Lippincott Williams & Wilkins. 2005).

A composition comprising the nanoparticles and carrier solution for the nanoparticles may have a pH that supports or promotes antimicrobial activity and continuous release of BPO, BC and/or BAC once applied to a surface or materials. The carrier solution may have a pH ranging from <3, 3, 4, 5, 6, 7, 8, 9, 10 or >10, preferable such a composition has a pH ranging from about 6, 7, or 8 for purposes of safety. The antimicrobial effectiveness of BPO, BC and/or BAC may increase when applied in combination with certain solvents such as, but not limited to, acetone, benzene, acetic acid, ethanol, triethanolamines, sodium hydroxide solutions, ethylenediamine tetra-acetate sodium, or any combination of organic solvents. Preferably, a solvent is selected that inhibits or prevents the release of BPO, BC and/or BAC during storage. A composition may be formulated as a pharmaceutical composition, a disinfectant, sanitizer, detergent, chelator, or antiseptic, suitable for reducing the numbers, growth rate, or viability of one or more types of microbes.

A composition incorporating the nanoparticles or coated nanoparticles disclosed herein may comprise carrier solution such as water, saline (e.g. normal saline), aqueous dextrose, glycerol, glycols, ethanol or the like) in a form of a solution or a suspension of nanoparticles.

In some embodiments, the nanoparticles or coated nanoparticles are admixed with dry ingredients or excipients, for example, to produce a powder or dry film.

The nanoparticle compositions disclosed herein may be formulated as creams, gels, foams, ointments, emulsions, powders, solutions, elixirs, lotions, suspensions, tinctures, pastes, foams, aerosols, irrigations, sprays, suppositories, swabs, moist- or non-moist woven or non-woven wipes, bandages, dermal patches or any other formulations suitable for topical administration. Such a composition may contain a bacteriostatic agent such as EDTA or EGTA.

A nanoparticle composition may be formulated for direct application to a surface or object, for example, by spraying or by production of an aerosol comprising the nanoparticles or coated nanoparticles disclosed herein. Such a composition may be formulated to evaporatively dry on a surface or object once applied.

Aerosols. Aerosol as provided herein includes products packaged under pressure and contain ingredients that are released upon activation of an appropriate valve system. Aerosols include all self-contained pressurized products, such as fine mists of spray or foam, that are emitted from a pressurized container containing a propellant, foams, or semisolid liquids. They may also be emitted by an unpressurized atomizer that is pressurized by a hand-operated pump rather than by stored propellant. In one embodiment, the aerosol comprises a container, a propellant, a concentrate containing an active ingredient, a valve (which may be a metered valve), and an actuator. The nature of these components determines characteristics such as delivery rate, foam density, and fluid viscosity. In another embodiment, the aerosol is a two-phase formulation comprising a gas and liquid. In another embodiment, the aerosol is a three-phase formulation comprising a gas, liquid, and suspension or emulsion of active ingredients. In this formulation, suitable excipients, such as wetting agents and/or solid carriers such as talc or colloidal silicas are included. In another embodiment, the propellant is liquefied or vaporized. In another embodiment, a solvent can be the propellant or a mixture of the propellant and co-solvents such as alcohol and polyethylene glycols. In another embodiment, the propellant is selected from the group comprising a spray, foam, or quick-breaking foam. In another embodiment, spray formulations are aqueous solutions in a container having a spray means, such as an atomizer or nebulizer. A spray may contain an aerosol of solid or encapsulated particles of a dry or wet BPO, BC, and/or BAC, composition in an atomized or aerosol liquid form.

Foams. In some embodiments, the disclosed compositions containing BPO, BC and/or BAC is delivered to an object or surface while in a foam state, such as stable foam, for example, that is produced with or without a propellant. In some versions, a foam is dispensed from a dispenser such as a propellant-free dispenser with pumping action to create the foam from a composition in a foamable carrier, and then applied directly to a surface or to a wipe or other substrate. Propellant-driving foam generators may also be used to deliver the composition in the form of a foam.

Active ingredients in a foam may be dispensed for subsequent placement on a dry wipe, a pre-moistened wipe, or other soft, flexible applicator (e.g., an object about 3-fingers wide or 4 to 10 cm wide) or other object to be used for application of the foam-based composition to the skin. The foam can be a non-propellant foam. A foam with a suitable stiffness of yield stress can be applied to the skin in any manner for sustained adherence and contact with the body. Examples of foam-based systems are described in U.S. Pat. No. 6,818,204, “Stable Foam for Use in Disposable Wipe,” issued to Lapidus on Nov. 16, 2004, herein incorporated by reference. The Lapidus patent involves the use of compatible surfactants, e.g., nonionic, anionic, amphoteric, for use in human hygienic products. The surfactant should be capable of forming a foam when mixed with air in a finger actuated, mechanical pump foamer. Such surfactants are said to include, without limitation, those which do not irritate mucous membranes such as polyethylene 20 cetyl ether (Brij 58)™, a nonionic surfactant; sodium lauroyl sarcosinate (Hamposyl L-30)™, sodium lauryl sulfoacetate (Lathanol LAL)™. and sodium laureth sulfate (Sipon ESY)™, anionic surfactants; lauramidopropyl betaine (Monateric LMAB™), an amphoteric surfactant, as well as polysorbate 20, TEA-cocoyl glutamate, disodium cocoamphodiacetate and combinations thereof. Typically, the surfactant is said to present in an amount from about 2% to about 35% by weight, or from about 5% to about 15% by weight.

At least one foam stabilizing agent may be present in some foamable embodiments. Suitable foam stabilizing agents may include, without limitation, natural or synthetic gums such as xanthan gum, polyalkylene glycols such as polyethylene glycol, alkylene polyols such as glycerine and propylene glycol and combinations thereof. Typically, the foam stabilizers may be present in an amount from about 0.10% to about 5%, or from about 2% to about 4%. In the Lapidus patent (U.S. Pat. No. 6,818,204), alkylene polyols are said to be typically employed in amounts from about 0.1% to about 10%, gums are employed in amounts ranging from about 0.05% to about 1%, and/or polyalkylene glycols are present in amounts ranging from about 0.05% to about 2%.

A foam may be produced using the F2 FINGER PUMP FOAMER™ manufactured by AirSpray International Inc. of Pompano Beach, Fla. Such a spring-loaded valve system operates without the use of gas propellants or the like. Upon actuation, precise amounts of air and liquid are mixed, and a foam capable of maintaining its structure for a substantial length of time is dispensed. In addition, the dispenser can deliver a variable amount of foam, thereby reducing waste of the wipe agent contained therein. Details of exemplary propellantless defoamers are described in U.S. Pat. No. 5,443,569, issued on Aug. 22, 1995, and U.S. Pat. No. 5,813,576, issued Sep. 29, 1998, herein incorporated by reference.

Embodiments of this technology include, but are not limited to the following.

A composition comprising benzoyl peroxide (“BPO”), benzyl chloride (“BC”), and/or benzalkonium chloride (BAC″) (or other organic peroxides) encapsulated in nanoparticles of polymethylmethacrylate (PMMA). In some embodiments, the benzoyl peroxide is encapsulated or the benzyl chloride is encapsulated. In other embodiments, the composition contains benzoyl peroxide and further comprises benzalkonium chloride. In some embodiments, the composition contains benzyl chloride and further comprises benzalkonium chloride.

In some embodiments, the composition as disclosed above is encapsulated in nanoparticles comprising PMMA that when applied to an external object or surface release an antimicrobial amount of BPO, BC and/or BAC over a period of 48 hours or more.

In other embodiments, the composition as disclosed above is encapsulated in nanoparticles comprising PMMA that range in average diameter from 50 to 500 nm.

In some embodiments, the composition as disclosed above contains 0.01 to 1.0 wt % benzoyl peroxide.

In other embodiments, the composition as disclosed above contains 0.01 to 1.0 wt % benzoyl chloride.

In some embodiments, the composition as disclosed above contains 0.01 to 1.0 wt % benzalkonium chloride.

The composition as disclosed above may be prepared or dispensed in the form of a spray or foam or as an aerosol.

In one embodiment, the composition as disclosed herein is prepared and dispensed from a container which comprises the composition, a propellant, and a nozzle.

Another embodiment of the invention is directed to a method for inhibiting the growth of a microorganism comprising contacting a microorganism with the composition of as disclosed herein. In some embodiments, the microorganism is a virus and said inhibiting inactivates or kills the virus. In other embodiments, the microorganism is a gram positive bacterium or a gram-negative bacterium. In other embodiments, the microorganism is a yeast or fungi; or is a parasite, protest, or other eukaryotic pathogen.

Another aspect of the invention is directed to a method for making the composition as disclosed herein comprising (i) dissolving PMMA in a solute containing a surfactant, (ii) adding benzoyl peroxide, benzyl chloride and/or benzalkonium chloride to the dissolved PMMA, (iii) adding a non-solvent to the solution of PMMA and BPO, BC and/or BAC for a time and under conditions suitable for nanoprecipitation of the composition containing PMMA and benzoyl peroxide, benzyl chloride or benzalkonium chloride, into nanoparticles.

Another aspect of the invention is directed to a method for making the composition as disclosed herein comprising (i) dissolving PMMA in a solute adding benzoyl peroxide and/or benzyl chloride to the dissolved PMMA, (ii) adding a non-solvent containing benzalkonium chloride in addition to a surfactant for a time and under conditions suitable for nanoprecipitation of the composition containing PMMA and benzoyl peroxide, benzyl chloride or benzalkonium chloride, into nanoparticles.

Another aspect of the invention is directed to a method for making the composition as disclosed herein comprising (i) dissolving PMMA in a solute adding benzoyl peroxide and/or benzyl chloride to the dissolved PMMA, (ii) adding a non-solvent (that is not miscible with the original solvent) containing benzalkonium chloride in addition to a surfactant for a time and under conditions suitable for nanoemulsion of the composition containing PMMA and benzoyl peroxide, benzyl chloride or benzalkonium chloride, into nanoparticles.

Another aspect of the invention is directed to a method for making the composition as disclosed herein comprising (i) dissolving benzoyl peroxide and/or benzyl chloride in a suitable solvent, (ii) adding a non-solvent containing benzalkonium chloride in addition to a surfactant for a time and under conditions suitable for nanomicelles of the composition containing benzoyl peroxide, benzyl chloride or benzalkonium chloride, into nanomicelles.

In some embodiments, the methods for making the composition as disclosed herein may employ at least one lab reactor, microfluidic chip, microject reactor, main spray dryer, or nanospray dryer.

Terminology. Terminology used herein is for the purpose of describing particular embodiments only and is not intended to be limiting of the invention.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all subranges subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology. As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1. A nanoparticle composition comprising benzoyl peroxide (“BPO”), benzyl chloride (“BC”), benzalkonium chloride (“BAC”) and/or another organic peroxide, encapsulated in a material comprising polymethylmethacrylate (PMMA) and, optionally, one or more carriers or excipients.

2. The composition of claim 1, wherein benzoyl peroxide is encapsulated.

3. The composition of claim 1, wherein benzyl chloride is encapsulated.

4. The composition of claim 1, which contains benzoyl peroxide and further comprises benzalkonium chloride.

5. The composition of claim 1, which contains benzyl chloride and further comprises benzalkonium chloride.

6. The composition of claim 1 that is encapsulated in nanoparticles comprising PMMA that when applied to an external object or surface release an antimicrobial amount of BPO, BC and/or BAC over a period of 48 hours or more.

7. The composition of claim 1 that is encapsulated in nanoparticles comprising PMMA that range in average diameter from 50 to 500 nm.

8. The composition of claim 1 that contains 0.01 to 1.0 wt % benzoyl peroxide based on the total volume of the nanoparticles.

9. The composition of claim 1 that contains 0.01 to 1.0 wt % benzoyl chloride based on the total volume of the nanoparticles.

10. The composition of claim 1 that contains 0.01 to 1.0 wt % benzalkonium chloride based on the total volume of the nanoparticles.

11. The composition of claim 1 in the form of a spray or foam.

12. The composition of claim 1 in the form of an aerosol.

13. A container comprising the composition of claim 1, a propellant, and a nozzle.

14. A surface or object treated with the composition of claim 1, wherein said surface or object comprises nanoparticles encapsulating the BPO, BC and/or BAC, or a mixture of nanoparticles encapsulating BPO and BC, BPO and BAC, or BC and BAC; wherein, optionally, the surface has been dried or evaporatively dried.

15. A method for inhibiting the growth of a microorganism comprising contacting a microorganism with the composition of claim 1.

16. The method of claim 15, wherein the microorganism is a virus and said inhibiting inactivates or kills the virus.

17. The method of claim 15, wherein the microorganism is a gram positive bacterium.

18. The method of claim 15, wherein the microorganism is a gram-negative bacterium.

19. The method of claim 15, wherein the microorganism is a yeast or fungi.

20. The method of claim 15, wherein the microorganism is a parasite, protist, or other eukaryotic pathogen.

21. A method for making the composition of claim 1 comprising (i) dissolving PMMA in a solute containing a surfactant, (ii) adding benzoyl peroxide, benzyl chloride and/or benzalkonium chloride to the dissolved PMMA, (iii) adding a non-solvent to the solution of PMMA and BPO, BC and/or BAC for a time and under conditions suitable for nanoprecipitation of the composition containing PMMA and benzoyl peroxide, benzyl chloride or benzalkonium chloride, into nanoparticles.

22. A method for making the composition of claim 1 comprising (i) dissolving PMMA in a solute adding benzoyl peroxide and/or benzyl chloride to the dissolved PMMA, (ii) adding a non-solvent containing benzalkonium chloride in addition to a surfactant for a time and under conditions suitable for nanoprecipitation of the composition containing PMMA and benzoyl peroxide, benzyl chloride or benzalkonium chloride, into nanoparticles.

23. A method for making the composition of claim 1 comprising (i) dissolving PMMA in a solute adding benzoyl peroxide and/or benzyl chloride to the dissolved PMMA, (ii) adding a non-solvent (that is not miscible with the original solvent) containing benzalkonium chloride in addition to a surfactant for a time and under conditions suitable for nanoemulsion of the composition containing PMMA and benzoyl peroxide, benzyl chloride or benzalkonium chloride, into nanoparticles.

24. A method for making the composition of claim 1 comprising (i) dissolving benzoyl peroxide and/or benzyl chloride in a suitable solvent, (ii) adding a non-solvent containing benzalkonium chloride in addition to a surfactant for a time and under conditions suitable for nanomicelles of the composition containing benzoyl peroxide, benzyl chloride or benzalkonium chloride, into nanomicelles.

25. A method for making the composition of claim 1, wherein said composition is fabricated using at least one lab reactor, microfluidic chip, microject reactor, main spray dryer, or nanospray dryer.
